(19) United States
(12) Patent Application Publication
(10) Pub. No.: US 2026/0351394 A1
(43) Pub. Date: Nov. 5, 2026

(54) SYSTEMS AND METHODS FOR A CLOUD-BASED SOFTWARE ARCHITECTURE WITH MUL Tl-TIER DEPLOYMENT AND API INTEGRATION

(71) Applicant: Easifi, Inc., Montclair, NJ (US)

(72) Inventor: Mohammed Rahman, Montclair, NJ (US)

(73) Assignee: Easifi, Inc., Montclair, NJ (US)

(21) Appl. No.: 19/663,398

(22) Filed: Apr. 30, 2026

Related U.S. Application Data
(60) Provisional application No. 63/797,131, filed on Apr. 30, 2025.

(51) Int. Cl. G16H 40/20

(52) U.S. Cl. CPC G16H 40/20

(57) ABSTRACT

A system includes a web-based and mobile-based platform designed to enhance communication and collaboration among dental professionals, laboratories, and third-party service providers. The system features a presentation layer with a user interface that displays case status, task lists, and patient information, enabling efficient case management. An application layer executes server-side logic to handle business processes, authentication, and role-based access control, ensuring secure and streamlined operations. The data layer incorporates a database management system for storing patient records, treatment plans, and transaction logs in a structured format. Additionally, a REST API facilitates interoperability with third-party service providers, allowing integration with treatment planning software, remote monitoring tools, and payment processing services. The system supports automated notifications, telehealth consultations, and scalable cloud-based infrastructure to optimize workflow efficiency. A role-based access control mechanism facilitates appropriate permission levels for different users, including dentists, consultants, laboratories, and technology providers, maintaining security and compliance.

### CROSS-REFERENCE TO RELATED APPLICATIONS

This application is a U.S. Non-Provisional Utility Patent Application entitled, “SYSTEMS AND METHODS FOR A CLOUD-BASED SOFTWARE ARCHITECTURE WITH MULTI-TIER DEPLOYMENT AND API INTEGRATION” which claims priority to co-pending U.S. Provisional Patent Application No. 63/797,131, filed on Apr. 30, 2025 entitled, “SYSTEMS AND METHODS FOR A CLOUD-BASED SOFTWARE ARCHITECTURE WITH MULTI-TIER DEPLOYMENT AND API INTEGRATION” the contents of which are hereby fully incorporated by reference.

### FIELD OF THE EMBODIMENTS

The present disclosure relates to digital dental case management systems, specifically to a platform that facilitates communication, collaboration, and workflow integration among dental professionals, laboratories, and service providers. The system is configured to facilitate orthodontic, oral appliances for sleep apnea, and restorative treatment planning by consolidating various tools and services into a unified interface, enhancing efficiency and interoperability in the dental industry.

### BACKGROUND OF THE EMBODIMENTS

In the field of orthodontics, dental treatment for sleep apnea, and restorative dentistry, collaboration among dental professionals, laboratories, and service providers is utilized for delivering patient care. However, many existing digital treatment planning and case management systems are often fragmented, proprietary, and restrictive. These closed-ended platforms, such as those offered by Align Technology, ClearCorrect, and 3Shape, limit flexibility by requiring providers to use their specific treatment planning software, aligner products, or in-house technicians. As a result, dental professionals face challenges in customizing treatment approaches to meet the diverse needs of their patients. For example, one problem in the industry is the lack of a singular, centralized platform that facilitates communication and collaboration among dentists, dental laboratories, and third-party service providers, such as treatment planning software companies, remote monitoring services, and consultants. Many current systems require dental professionals to navigate multiple platforms and software tools, leading to inefficiencies, increased costs, and workflow disruptions. This fragmented approach often results in miscommunications, delays in treatment planning, and limited adaptability to individual patient needs.

Moreover, existing closed-ended platforms impose rigid pricing structures, often requiring dental professionals to pay per case or per service, without the flexibility of a subscription-based model. This lack of pricing transparency and customization can make high-quality orthodontic care more expensive and less accessible to both providers and patients. In an embodiment, current systems do not always provide adequate integration with third-party solutions, further restricting the ability of dental professionals to optimize treatment plans based on their clinical expertise and patient preferences. Given the unique nature of each patient's needs, budget, and case complexity, there is a demand for an improved, centralized solution that consolidates one or more tools, such as treatment planning software, aligner and device selection, and progress monitoring, into a single, user-friendly platform. A system that facilitates dental professionals to design, create, and monitor aligner and sleep apnea cases with greater autonomy and interoperability is needed to enhance efficiency, reduce costs, and ultimately improve patient outcomes.

### SUMMARY OF THE EMBODIMENTS

The digital platform is implemented for facilitating communication, collaboration, and case management among dental professionals, laboratories, and third-party service providers. More particularly, the disclosure pertains to a web-based and mobile-based system that integrates treatment planning, remote monitoring, and other dental service functionalities into a centralized architecture. The system is implemented using a multi-tier architecture to facilitate scalability, modularity, and efficient data processing. The architecture comprises a presentation layer, an application layer, and a data layer. The presentation layer is responsible for rendering the user interface and providing access to the system's functionalities through web-based and mobile-based applications. The application layer executes business logic, processes user requests, and facilitates interactions between the user interface and underlying data structures. The data layer manages data storage, retrieval, and integrity, facilitating secure and structured access to patient records, case files, and treatment plans.

In an embodiment, the system utilizes various technologies to enable efficient operation. The front-end framework employs HTML5, CSS3, JavaScript, and React.js to create a responsive and user-friendly interface. The back-end infrastructure is developed using Node.js and Express.js, providing a framework for processing data, managing authentication and authorization, and executing business logic. The data storage component is implemented using MongoDB, a NoSQL database that facilitates scalable data persistence and retrieval. The system includes multiple integrated software components to employ operation. The user interface component provides an interactive dashboard displaying case status, task lists, educational videos, and collaboration tools for dental professionals. The server-side logic component handles authentication, role-based access control, and execution of treatment workflows. The database management component facilitates secure storage of patient data, case details, and transaction logs, maintaining compliance with industry regulations. The REST API component facilitates communication between the front-end application and the server-side processes, allowing interoperability with third-party service providers such as treatment planning software, remote monitoring tools, and consultation services.

In an embodiment, the system employs a stable execution environment with defined hardware and software specifications. For example, the system includes at least 2 GB of RAM, a 1 GHz processor, and 500 MB of available storage space. The software requirements for the mobile-based application include support for Java 8 or higher and MySQL 5.7 or higher. The system undergoes a structured testing process to validate functionality, security, and performance. The testing methodology includes unit testing to verify individual components, API testing to facilitate integration between services, and system integration testing to validate end-to-end workflows. In an embodiment, regression testing is performed to maintain stability across updates, while black-box testing facilitates that user interactions align with expected outcomes. The final validation phase includes mass user testing to assess system performance under real-world conditions. The deployment of the system utilizes a cloud-based hosting infrastructure to facilitate scalability and availability. The deployment process involves configuring the cloud environment, deploying application code, setting up the database and data storage systems, and configuring API endpoints to establish connectivity between system components. The cloud-based implementation allows for real-time updates, remote accessibility, and efficient resource management.

This disclosure presents a structured technical overview of a digital case management and collaboration platform configured for dental professionals. The system integrates multiple technologies to streamline treatment planning, remote monitoring, and communication among stakeholders in the dental industry. By employing a scalable and interoperable architecture, the platform enhances workflow efficiency, reduces operational costs, and improves patient care.

### BRIEF DESCRIPTION OF THE DRAWINGS

The present disclosure may be better understood, and its numerous features and advantages made apparent to those skilled in the art by referencing the accompanying drawings. The use of the same reference symbols in different drawings indicates similar or identical items.

FIG. 1 is a block diagram illustrating a system, according to some embodiments of the present disclosure.

FIG. 2 is a block diagram further illustrating the system from FIG. 1, according to some embodiments of the present disclosure.

FIG. 3 is a flowchart illustrating a method for facilitating communication and collaboration in dental treatment planning, according to some embodiments of the present disclosure.

FIG. 4 is a block diagram illustrating a cloud-based dental case management system, according to some embodiments of the present disclosure.

FIG. 5 is a block diagram illustrating a computer-implemented platform, according to some embodiments of the present disclosure.

FIG. 6 is a block diagram further illustrating the computer-implemented platform from FIG. 5, according to some embodiments of the present disclosure.

FIG. 7 is a block diagram further illustrating the computer-implemented platform from FIG. 5, according to some embodiments of the present disclosure.

FIG. 8 is a block diagram further illustrating the computer-implemented platform from FIG. 5, according to some embodiments of the present disclosure.

FIG. 9 is a flowchart illustrating a method for processing payment information in a case management system, according to some embodiments of the present disclosure.

FIG. 10 is a block diagram illustrating a system, according to some embodiments of the present disclosure.

FIG. 11 is a flowchart illustrating a method, according to some embodiments of the present disclosure.

FIG. 12 is a block diagram of an example payment platform within the system, according to some embodiments of the present disclosure.

### DETAILED DESCRIPTION OF THE EMBODIMENTS

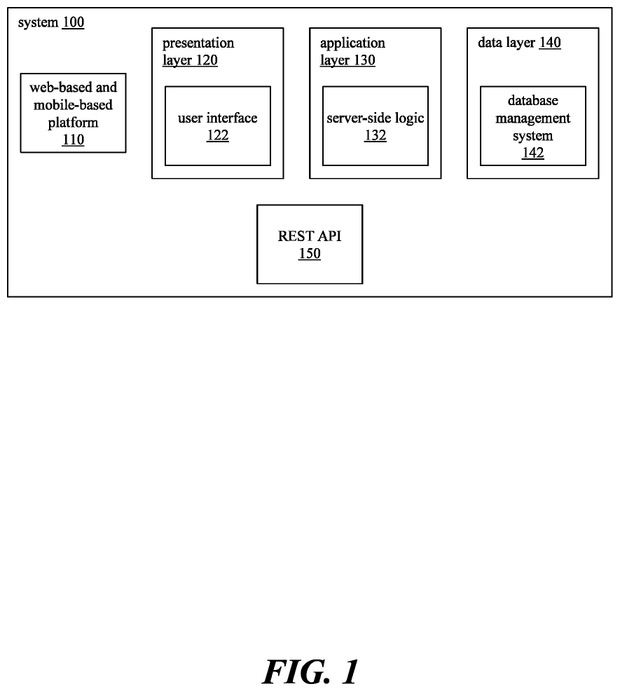

FIGS. 1-12 illustrate example methods and systems for implementing Easifi, a Business-to-Business (B2B) online marketplace configured to empower dental professionals with technology that facilitates the process of delivering esthetic and healthy smiles as well as dental treatment of obstructive sleep apnea. The system is configured to enhance patient care while reducing expenses for dental professionals. Easifi's success is directly tied to the success of the dental professionals using the platform. The platform is crafted to equip one or more users with one or more tools, ensuring they can provide the best possible care for their patients. Since every patient has unique needs, budgets, and case complexities, Easifi simplifies case management by consolidating various tools, such as treatment planning software, aligner and oral appliance types, and progress monitoring, all within one user-friendly platform. This allows dental professionals the freedom to design, create, and monitor aligner cases and oral appliances tailored to both their practice and their patients.

The external users of the platform include dentists, consultants, labs, and tech companies. Dentists are the primary users, creating and managing cases and adding other users and services. Consultants, who are also dentists, have more limited rights and are assigned to assist in cases, adding services with the dentist's approval. Labs, as service providers, can be added to cases by either dentists or consultants, and they can add services from other labs or tech companies, subject to dentist approval and payment for these services. Tech companies, also service providers, are added by dentists or consultants but cannot add any services to a case. Internally, Easifi is managed by admins and owners, who oversee all external users, service providers, transactions, and the homepage of the platform. The technical aspects are handled by a Super Admin from Medina Tech.

The platform features include authentication, case management, service management, file management, user profiles, search and filter options, sending and accepting requests, payment processing, case status tracking, messaging, and more. External users can access the platform through provided UI links, although updates are still pending for the tech company UI, registration UI, and payment UI. The project also includes a backlog of features to be developed, such as the ability for users (dentists, labs, consultants, and tech companies) to register on the platform with specific fields depending on their role. These roles also have specific authentication and profile management functionalities, such as password resets, profile updates, and the ability to manage public profiles.

For case management, dentists can create, update, and delete cases, with mandatory fields including patient details. Only dentists can delete a case, and they can control the addition of services by consultants or labs, with approval required for services added by others. Users can upload, download, and manage case files and services, ensuring that case progress is appropriately tracked and managed. The platform also allows users to manage requests, search and filter case lists, and navigate through past cases. Various search filters enable users to find relevant information based on case type, location, pricing, and service providers. This comprehensive system supports case and service management, making the platform an indispensable tool for dental professionals.

FIG. 1 is a block diagram illustrating an overview of a system 100, according to certain embodiments of the present disclosure. In these embodiments, the system 100 includes several layers and components configured to facilitate dental treatment planning. In an embodiment, the system includes a presentation layer 120, an application layer 130, a data layer 140, and a REST API 150 that facilitates integration with third-party service providers. In an embodiment, the system features a web-based and mobile-based platform 110 that supports communication and collaboration among dental professionals, laboratories, and third-party service providers. In further detail, the presentation layer 120 includes a user interface 122 configured to display patient information, case status, and task lists. The application layer 130 contains server-side logic 132 responsible for managing business processes, user authentication, and role-based access control. The data layer 140 houses a database management system 142 for storing patient records, treatment plans, and transaction logs.

The user interface 122 is implemented using modern web technologies such as HTML5, CSS3, JavaScript, and React.js. The server-side logic 132 is powered by Node.js and Express.js, while the database may be managed using MongoDB to store both structured and unstructured data. The system also supports an electronic signature feature for digitally approving treatment plans. The REST API 150 allows integration with other treatment planning software, remote monitoring systems, and payment processing services. The system also includes billing and invoicing processors for processing payments between dental professionals and service providers. The platform 110 may further support multi-language interfaces, enhancing accessibility for international users.

In an embodiment, the system utilizes a user interface implemented using modern web technologies, including HTML5, CSS3, JavaScript, and React.js. These technologies work together to create a responsive and user-friendly experience, allowing dental professionals to easily interact with the platform. HTML5 and CSS3 provide the foundational structure and styling, while JavaScript and React.js enable dynamic functionality, ensuring that the interface remains interactive and capable of real-time updates.

In an embodiment, for backend operations, the system leverages Node.js and Express.js. Node.js, a JavaScript runtime, allows the system to efficiently handle multiple simultaneous requests, ensuring smooth performance even under high usage. Express.js, a web application framework for Node.js, simplifies the process of routing and managing HTTP requests, making the backend more streamlined and easier to maintain.

In an embodiment, to store and manage dental case data, the system employs MongoDB, a NoSQL database. MongoDB's flexibility in handling both structured and unstructured data makes it an ideal choice for managing complex data types, such as 3D dental scans, intraoral images, and X-ray data, alongside traditional patient information like demographics and treatment plans. This capability facilitates that the system can scale to accommodate large volumes of data as the dental practice grows.

In an embodiment, the system also includes an electronic signature feature, enabling dental professionals to approve treatment plans digitally. This feature streamlines the approval process, eliminating the need for paper-based signatures and allowing for faster documentation of patient consent. For example, once a treatment plan is reviewed with a patient, the dentist can securely sign it electronically, ensuring that all approvals are recorded and verifiable.

In an embodiment, a REST API is integrated into the system to facilitate communication with other dental technologies and services. The API facilitates the system to interact with treatment planning software, remote monitoring systems, and payment processing services. For instance, the API might allow the system to share patient data with specialized orthodontic software or receive real-time updates from remote monitoring devices tracking a patient's treatment progress.

In an embodiment, the platform also includes a billing and invoicing processor to handle financial transactions between dental professionals and service providers. This feature simplifies payment processing, automatically generating invoices and managing payment collection. For example, after completing a treatment, the system can automatically create an invoice and process payment through an integrated gateway, reducing the administrative burden on dental staff.

In an embodiment, to facilitate case management, the system uses color-coded status indicators to track the progress of each treatment. These indicators visually represent the stages of treatment, such as “pending,” “in progress,” and “completed,” helping dental professionals quickly assess the status of ongoing cases. For example, a dentist can easily determine whether a treatment is awaiting approval, actively being carried out, or has been completed.

In an embodiment, the platform is configured with international accessibility in mind, supporting multi-language interfaces to accommodate users around the world. This feature facilitates that dental professionals in different countries can interact with the system in their native language, making it easier for global practices to adopt the platform without facing language barriers.

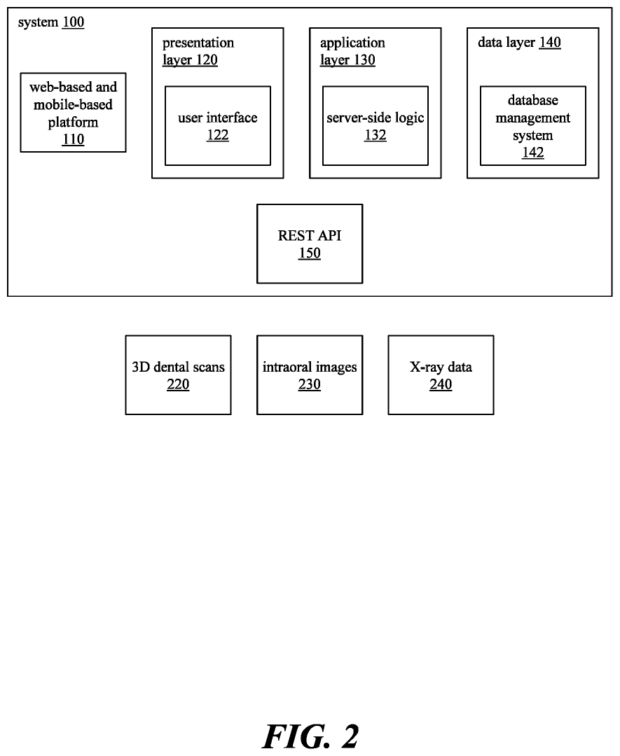

FIG. 2 is a block diagram that expands upon the system 100 from FIG. 1, providing further details about the integration of patient records within the overall system architecture. This diagram illustrates how various components of the system interact to facilitate efficient case management and smooth data flow. In some embodiments, patient records are central to the system's operation and may include a variety of dental data, such as 3D dental scans 220, intraoral images 230, and X-ray data 240. These records are stored in a secure format to maintain patient confidentiality and comply with data protection regulations such as HIPAA.

For example, 3D dental scans 220 can be used to create detailed models of a patient's teeth and jaw structure, which are crucial for treatment planning, particularly in complex procedures like orthodontics or implantology. Intraoral images 230 capture real-time pictures of the patient's mouth, allowing dental professionals to monitor conditions such as cavities, gum disease, or oral lesions. X-ray data 240 offers valuable insights into bone structure, tooth alignment, and the presence of underlying issues that may not be visible to the naked eye. These patient records are securely stored in the system's database, which can be accessed by dental professionals, such as dentists and orthodontists, through the user interface of the platform. In an embodiment, the system facilitates sharing of these records between the dental team, including consultants, dental laboratories, and third-party service providers, ensuring that everyone involved in the treatment plan has access to the necessary information.

The integration of patient records with the system allows for real-time updates to the user interface, providing dental professionals with the latest case information. For example, when a new 3D dental scan is uploaded or a treatment plan is modified, the changes are immediately reflected in the system, ensuring all stakeholders have the most current data available for decision-making. In some embodiments, secure APIs (Application Programming Interfaces) may be used to enable interoperability with other medical or dental systems, allowing for the exchange of patient records and case data across different platforms. These integrations help enhance the overall efficiency of the dental case management process, reducing errors and improving patient care outcomes.

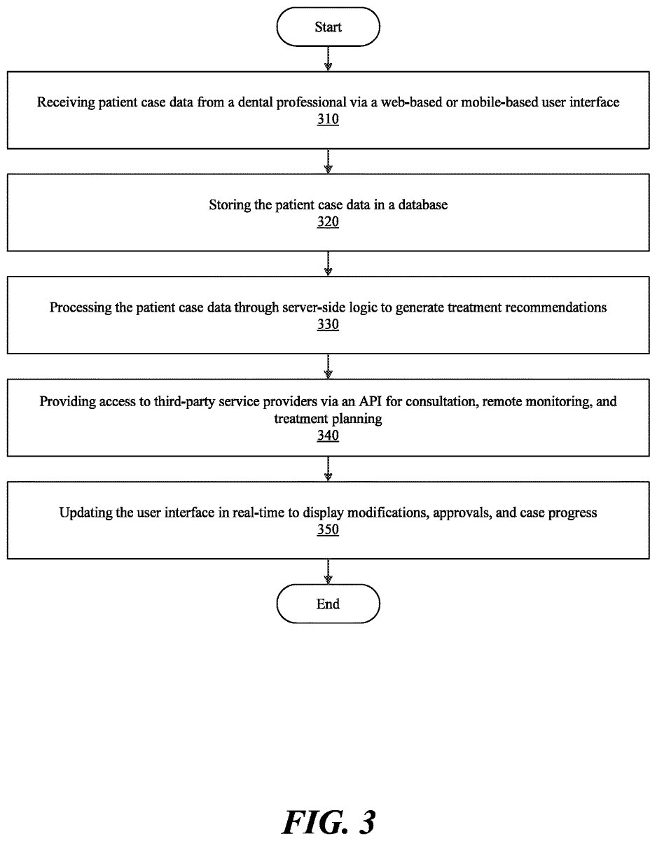

FIG. 3 is a flowchart depicting a method for enhancing communication and collaboration in dental treatment planning, according to certain embodiments. The method begins with block 310, where patient case data is received from a dental professional via a web-based or mobile interface. In block 320, the system stores this data in a database for future reference. Block 330 involves processing the data through server-side logic to generate treatment recommendations. Next, at block 340, third-party service providers are granted access to the case data via the API, enabling collaboration on consultation, remote monitoring, and treatment planning. Block 350 focuses on updating the user interface in real time to reflect any modifications, approvals, or progress in the case. The method may also involve automatic assignment of cases to dental laboratories based on predefined criteria, along with real-time feedback from remote monitoring systems that track patient progress through wearable devices.

As noted above, the process begins in block 310, where patient case data, such as dental history, X-rays, and 3D scans, is uploaded by a dental professional using either a web-based or mobile interface. For example, a dentist might upload a patient's intraoral images and X-ray data for a comprehensive view of the patient's dental health, ensuring all relevant information is captured in one place.

In block 320, the system stores the data securely in a database for future reference. This facilitates that all patient data is organized and readily accessible for future consultations or follow-up treatments. As the data is stored, it is indexed and categorized based on patient ID, treatment type, and other relevant factors, facilitating efficient retrieval when needed. For instance, a dental hygienist reviewing a patient's records for routine cleaning can quickly pull up the latest treatment data.

Block 330 involves processing the data through server-side logic, where algorithms analyze the case data to generate personalized treatment recommendations. For instance, based on the uploaded 3D dental scans and X-rays, the system may suggest specific orthodontic treatments or recommend restorative procedures tailored to the patient's needs. This step facilitates that dental professionals have evidence-based, customized guidance to support their decision-making process.

In block 340, third-party service providers, such as dental specialists or laboratories, gain access to the case data through a secure API. This API facilitates collaborative efforts between dental professionals and service providers, such as remote consultations or consultations with orthodontists who may offer input on the treatment plan. For example, an orthodontist in another location can remotely evaluate the patient's case and provide expert recommendations, enhancing collaboration across distances.

Block 350 focuses on updating the user interface in real time to reflect any modifications, approvals, or progress in the case. As the treatment plan evolves or is updated, whether through professional input, patient feedback, or new diagnostic information, the system immediately reflects these changes on the dental professional's interface. For example, if a treatment plan is approved by a patient, the interface will update to show the plan's status as “approved,” ensuring that everyone involved in the case is kept informed.

In an embodiment, the method may include automatically assigning cases to dental laboratories based on predefined criteria, such as the need for crowns, bridges, or dentures. This automated process streamlines the workflow and facilitates that lab work is initiated without delay, reducing the potential for treatment bottlenecks. Furthermore, real-time feedback from remote monitoring systems, such as wearable devices or sensors, can track patient progress during treatment. For instance, a patient undergoing orthodontic treatment may use a connected device that tracks the movement of their teeth and provides updates, which are then automatically fed into the system, allowing dental professionals to monitor progress and adjust treatment as necessary. This level of continuous, data-driven feedback enhances the overall efficiency and success of the treatment plan.

In an embodiment, the system automatically assigns cases to dental laboratories based on predefined criteria. For example, if a patient requires the fabrication of a dental crown, the system can automatically assign the case to a laboratory with expertise in crown production based on the complexity of the case or the laboratory's specialization. The assignment process helps streamline workflows, ensuring that the right laboratory is selected and that the treatment process progresses without delays.

In an embodiment, remote monitoring services are integrated into the system, providing real-time feedback on patient progress through wearable devices. For example, a patient wearing a smart dental aligner with built-in sensors can transmit data to the system, allowing the dentist to monitor the alignment process remotely. This continuous feedback facilitates dental professionals to make timely adjustments to the treatment plan, enhancing the accuracy and effectiveness of the treatment.

In an embodiment, the system includes an automated case validation feature that cross-references treatment plans against predefined clinical guidelines. This validation process facilitates that the proposed treatment aligns with best practices and established protocols. For instance, if a treatment plan for a patient with periodontal disease does not meet the recommended criteria for conservative management, the system may flag the plan for review, prompting the dental professional to reconsider or modify the treatment approach before proceeding. This automated validation helps maintain consistency in treatment quality and facilitates that patients receive appropriate care.

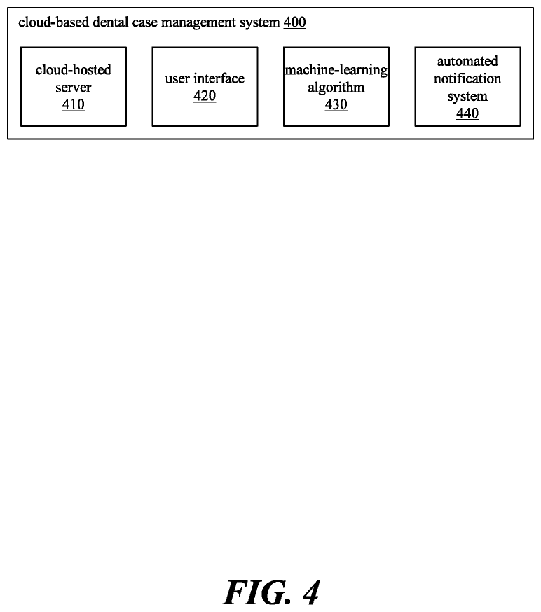

FIG. 4 is a block diagram illustrating a cloud-based dental case management system 400, according to certain embodiments. This system includes a cloud-hosted server 410, which manages application logic and stores patient data. A machine-learning algorithm 430 analyzes patient treatment history to recommend optimized treatment paths, and an automated notification system 440 sends alerts to dental professionals regarding case updates and deadlines. The system supports telehealth consultations between dental professionals and patients, enhancing the ability to manage cases remotely. In an embodiment, the cloud-hosted server 410 scales automatically to handle varying numbers of active cases and user loads. The automated notification system 440 also features role-based access control, ensuring that only authorized individuals can access sensitive data. Notifications can be sent via SMS, email, or in-app alerts. The cloud-based dental case management system 400 may also include a user interface 420 providing access to treatment plans, aligner and oral appliance design, and remote monitoring tools.

As noted above, a cloud-based dental case management system 400 is configured to enhance the efficiency and collaboration of dental professionals in managing patient treatment cases. The system includes a cloud-hosted server 410 that functions as the central hub for managing application logic and storing patient data. The server facilitates access to case information, enabling dental professionals to retrieve patient records and treatment plans from any location, thus improving accessibility and ensuring that the system can scale dynamically to accommodate varying numbers of active cases and user loads. For example, during peak periods, such as when multiple dental professionals are accessing the system to review cases, the cloud infrastructure can automatically allocate additional resources to maintain optimal performance.

The system implements a machine-learning algorithm 430 that analyzes a patient's treatment history to provide tailored treatment recommendations. For instance, based on a patient's previous dental procedures, the algorithm can suggest the most effective treatment paths for their current condition, such as recommending the use of specific materials or procedures that have been proven to yield the best outcomes for similar cases. This data-driven approach helps dental professionals make more informed decisions, improving patient care and reducing the likelihood of complications.

The cloud-based dental case management system can implement a variety of machine learning algorithms to enhance patient care, optimize treatment planning, and improve overall system efficiency. Classification algorithms such as decision trees and support vector machines can be used to categorize patient cases based on their severity or complexity, enabling dental professionals to prioritize cases and recommend tailored treatments. For example, a decision tree can analyze a patient's medical records, X-rays, and previous treatments to predict the likelihood of conditions like cavities or gum disease, offering specific treatment suggestions based on the predicted classification.

In an embodiment, one or more regression algorithms, including linear and polynomial regression, can be utilized to forecast continuous outcomes, such as recovery time following dental procedures. For instance, a regression model might predict a patient's recovery time based on variables like age, oral health status, and treatment complexity, allowing dental professionals to provide more accurate treatment timelines and manage patient expectations effectively.

In an embodiment, one or more clustering algorithms, like K-Means, can group patients with similar characteristics, helping to identify patterns and optimize treatment protocols for different patient segments. For example, the system could segment patients based on factors like age, gender, and health conditions, allowing for the development of tailored treatment plans for each group. Similarly, recommendation systems using collaborative or content-based filtering can suggest optimal treatments, dental products, or equipment based on patient history and outcomes from similar cases, thereby improving the quality of care.

In an embodiment, one or more natural language processing (NLP) algorithms can analyze unstructured data, such as patient notes or case summaries, to extract valuable information and provide context-aware recommendations or alerts. For example, NLP could be used to identify potential treatment concerns or symptoms from free-text records, enabling the system to flag relevant cases for further review. Anomaly detection algorithms can identify unusual patterns in patient data, such as irregular recovery trends, and alert dental professionals if further intervention is needed.

In an embodiment, one or more deep learning algorithms, including, but not limited to, convolutional neural networks (CNNs), can be applied to analyze dental images, such as X-rays and intraoral photographs, to detect conditions like cavities, fractures, or oral cancer. These models can assist dental professionals by providing automated diagnostic insights and early warnings of potential issues. In an embodiment, time-series forecasting algorithms, such as Long Short-Term Memory (LSTM) networks, can predict future events, such as patient follow-up visits or treatment progress, helping dental practices optimize scheduling and resources.

In an embodiment, one or more machine learning algorithms allow the dental case management system to deliver personalized, data-driven treatment plans, improving patient outcomes, reducing errors, and/or streamlining dental practice operations.

The automated notification system 440 facilitates that dental professionals are kept up-to-date on case developments and deadlines. This system generates alerts for case updates, such as when a treatment plan is ready for approval or when a scheduled consultation is approaching. Notifications can be delivered via multiple channels, including SMS, email, or in-app alerts, making it easy for dental professionals to stay informed regardless of their location. For example, if a treatment plan update is approved by a specialist, the system will send an automatic alert to the primary dentist, indicating that they can proceed with the next steps in the treatment process.

Role-based access control (RBAC) is a component of the notification system, ensuring that only authorized users can access sensitive patient data. For example, a dental assistant may have access to patient records and treatment plans, but a specialist or administrator may have additional privileges to modify treatment recommendations or approve changes to a case. This security feature helps protect patient privacy and facilitates compliance with healthcare regulations such as HIPAA.

Moreover, the system supports telehealth consultations, which allows dental professionals to conduct remote consultations with patients. For example, a dentist can assess a patient's oral health through a video call, review X-rays or intraoral images, and provide treatment recommendations all within the system. This capability significantly expands the accessibility of dental care, particularly for patients in remote areas or those unable to visit the clinic in person. The integration of telehealth capabilities with the cloud-based platform helps to streamline communication between patients and professionals, improving patient outcomes by enabling timely interventions.

In an embodiment, the system enables telehealth consultations between dental professionals and patients, allowing real-time video conferencing, remote diagnosis, and treatment discussions. For example, a dentist can review a patient's intraoral images and X-rays uploaded to the platform and provide recommendations without requiring an in-person visit. This feature is particularly beneficial for follow-up assessments, second opinions, and consultations with specialists in different locations.

In an embodiment, the cloud-hosted server dynamically adjusts its resources based on the number of active cases and user demand, ensuring optimal system performance and reliability. For instance, if a surge of users simultaneously access the platform to upload patient records or conduct virtual consultations, the server automatically allocates additional computational power to prevent slowdowns or downtime. Conversely, during periods of low activity, resources are scaled down to optimize cost efficiency without compromising service quality.

In an embodiment, a role-based access control processor governs data security by assigning different permission levels to various users, ensuring that sensitive patient information remains protected. Dentists may have full access to patient records and treatment histories, while dental laboratories can only view relevant case details needed for prosthetic fabrication or other lab work. Meanwhile, consultants, such as orthodontic specialists, may receive limited access to analyze specific case files and provide recommendations without the ability to modify treatment plans.

In an embodiment, the machine-learning algorithm is trained on historical patient treatment data, refining its predictive accuracy over time to enhance decision-making. For example, by analyzing thousands of past treatment records, the algorithm can identify patterns that suggest optimal recovery timelines for different procedures, predict potential complications based on patient history, and recommend the most effective treatment plans. As new data is continuously incorporated, the algorithm improves its ability to assist dental professionals in making evidence-based clinical decisions.

In an embodiment, the system delivers notifications through multiple communication channels, including SMS, email, and in-app alerts, ensuring timely updates for all users. For example, a dentist may receive an in-app notification about a pending case approval, while a laboratory technician is alerted via email when a new order is assigned. Patients may receive SMS reminders for upcoming virtual consultations or follow-up appointments, enhancing engagement and adherence to treatment plans. These notification methods improve workflow efficiency and facilitate that all stakeholders stay informed in real time.

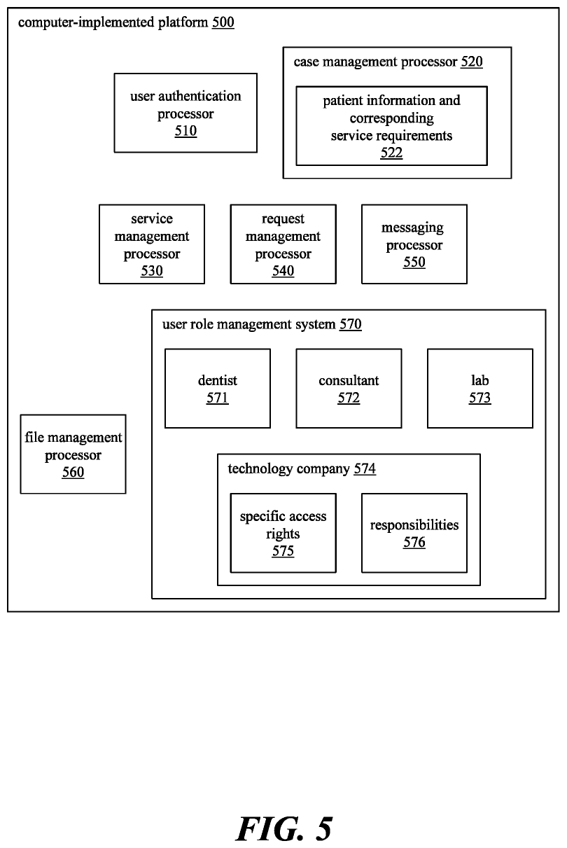

FIG. 5 is a block diagram depicting a computer-implemented platform 500, which provides an example case management system for dental professionals. The platform includes a messaging processor 550 that facilitates communication between users related to specific dental cases, and a user role management system 570 that defines user roles, such as dentists, consultants, and laboratories. The user authentication processor 510 handles user registration, login, and password management. The case management processor 520 allows users to create, update, and view dental cases, as well as track service progress and case statuses. The service management processor 530 facilitates users to manage services linked to dental cases, including adding, modifying, or approving services. In addition, the request management processor 540 facilitates users to send and respond to service requests. The file management processor 560 allows users to upload, download, and delete files associated with dental cases. The platform may also feature a verification processor to validate credentials such as a National Provider Identifier (NPI) and license number during registration.

The case management processor 520 may store and organize patient information along with corresponding service requirements 522, ensuring that each dental case is managed. For example, a patient's case file may include their medical history, 3D dental scans, X-rays, intraoral images, and a list of required treatments, such as crowns, implants, or orthodontic procedures. In an embodiment, service requirements can outline specific needs, such as materials for dental prosthetics, turnaround times for lab work, or follow-up appointment schedules. The user role management system 570 assigns different access levels and responsibilities to various stakeholders within the platform 500. This system includes one or more dentists 571, one or more consultants 572, one or more dental laboratories 573, and/or one or more technology companies 574, each with role-predetermined permissions. For instance, a dentist may have full access to patient records, allowing them to update treatment plans, approve lab work, and communicate with specialists. A consultant, such as an orthodontist or periodontist, may have restricted access limited to reviewing case files and providing expert recommendations. Laboratories can view case details relevant to prosthetic fabrication but cannot modify patient records.

In an embodiment, the technology company 574 has one or more predetermined access rights 575 and responsibilities 576 within the platform. For example, it may oversee system maintenance, provide customer support for platform users, and facilitate compliance with data security regulations. In an embodiment, the tech company may manage software updates, troubleshoot technical issues, and facilitate integrations with third-party applications, such as digital imaging tools or payment processing systems. These role-based access controls enhance security and streamline workflow efficiency by ensuring that each user interacts with only the information necessary for their role.

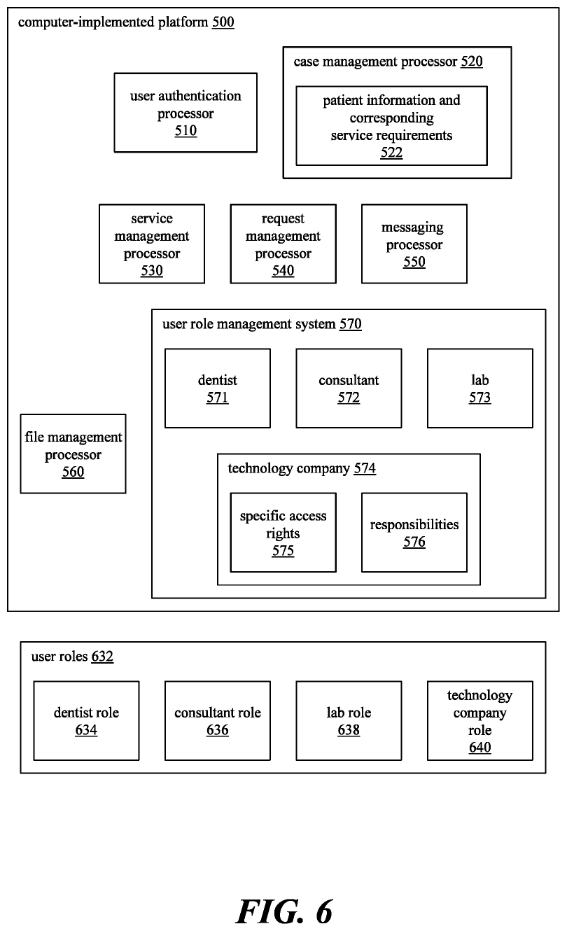

FIG. 6 expands on the user role management system 570 from FIG. 5. It defines specific roles and permissions within the platform. In some embodiments, the user roles 632 may include a technology company role 640 may allow users to provide services but not modify them without dentist approval. The dentist role 634 facilitates users to manage dental cases, approve services, and access patient details. The consultant role 636 allows users to assist with cases, add services, and request approval from the dentist to modify services. The lab role 638 lets users add services to cases, request approval, and manage associated case files.

As noted above, for example, the technology company role 640 enables users to provide technical services, such as maintaining the platform, offering customer support, or integrating third-party tools like digital imaging software. However, these users cannot modify patient treatment plans or case details without explicit approval from a dentist. This restriction facilitates that clinical decisions remain under the control of dental professionals. In an example, the dentist role 634 grants users' full management capabilities over dental cases. A dentist can initiate and approve treatment plans, access and update patient records, and authorize lab work. For instance, if a patient requires a crown, the dentist can review intraoral scans, prescribe the required restoration, and approve the case before sending it to the lab. Dentists can also communicate with consultants and laboratories directly through the platform to streamline the case workflow. In an example, the consultant role 636 is configured for specialists, such as orthodontists or periodontists, who assist in specific cases. Consultants can review patient data, provide expert recommendations, and suggest additional services, such as periodontal treatments or aligner therapy. However, they cannot modify treatment plans or perform services without requesting approval from the dentist. For example, a consultant may recommend an alternative treatment for a patient's misalignment but must obtain dentist approval before implementing changes. IN an example, the lab role 638 allows dental laboratories to contribute to case management by adding relevant services, such as designing crowns, bridges, or dentures. Lab users can upload case files, request necessary patient data (such as impressions or digital scans), and submit service completion reports. However, any modifications to the treatment plan, such as changing the type of material used for a prosthetic, require dentist approval. For example, if a lab technician identifies that a different ceramic material would be more suitable for a crown, they must submit a request for approval before proceeding with the change.

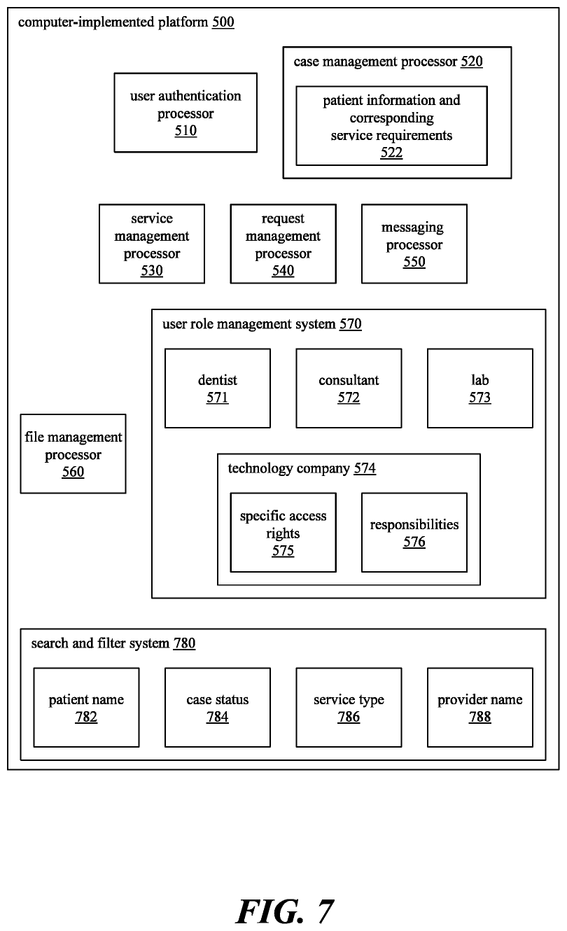

FIG. 7 further elaborates on the search and filter system 780 integrated into the platform 500. This system facilitates users to search for dental cases and filter results based on various criteria, such as patient name 782, case status 784, service type 786, and provider name 788. For instance, the patient name filter 782 allows users to search for specific patients by entering full or partial names. This feature is useful when a dentist needs to quickly access a patient's treatment history or upcoming procedures without manually browsing through a long list of cases. For example, entering “Smith” in the search bar would return all cases associated with patients having that last name. The case status filter 784 enables users to sort cases based on their progress, such as “Pending Review,” “In Progress,” “Awaiting Approval,” or “Completed.” A dentist may use this filter to prioritize urgent cases that require immediate approval, while a lab technician might filter for cases marked as “Awaiting Lab Work” to focus on active tasks. The service type filter 786 categorizes cases by treatment procedures, such as crowns, implants, root canals, or orthodontic aligners. This functionality helps streamline workflows by allowing specialists to focus on cases relevant to their expertise. For example, an orthodontist can filter cases to view only aligner treatments, ensuring they are working on relevant cases. The provider name filter 788 enables users to search for cases associated with specific dental professionals, such as referring dentists, consultants, or laboratories. A lab technician might use this feature to find all cases assigned by a particular dentist, while a specialist consultant can filter cases linked to a specific practice for follow-up. Additional filtering options may include date range selection, allowing users to find cases created, modified, or completed within a specific timeframe, or urgency level filtering, which highlights cases requiring immediate attention. By implementing these search and filter features, the system enhances efficiency and organization, reducing time spent on administrative tasks and enabling dental professionals to focus on patient care.

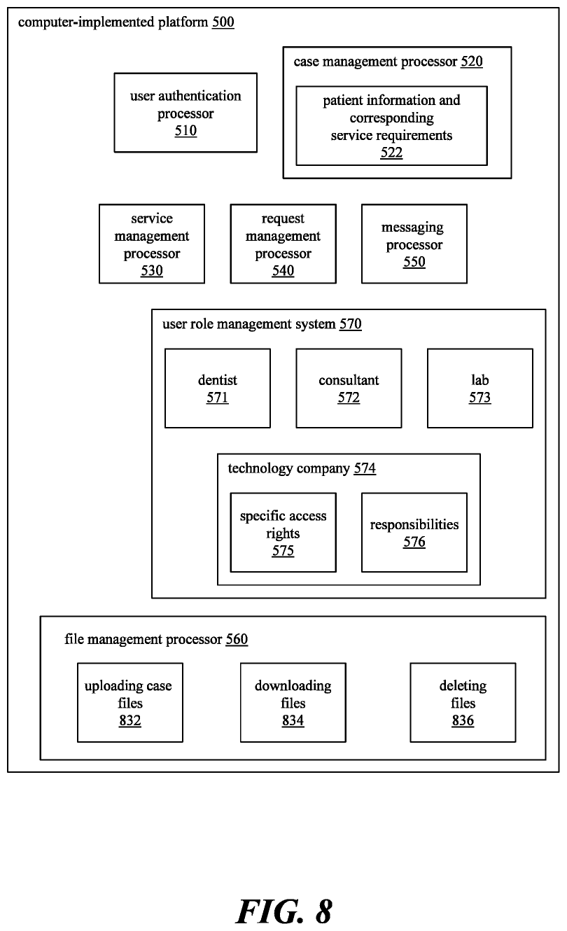

FIG. 8 illustrates the file management processor 560 from FIG. 5 in greater detail. It allows users to manage documents related to a dental case, including uploading, downloading, and deleting files as necessary. The file management processor 560 provides users with a comprehensive system for handling documents associated with a dental case, ensuring secure and efficient management of case-related files. Users can upload case files 832, such as digital treatment plans, 3D dental scans, intraoral images, and X-ray reports. For example, a dentist may upload a patient's panoramic X-ray to provide a consultant with a complete view of the case, or a lab technician may submit a 3D scan of a dental impression for review before fabricating a crown. The downloading files feature 834 allows authorized users to retrieve documents for offline access or further analysis. A consultant reviewing a treatment plan may download the relevant case files for in-depth evaluation, or a dental professional may retrieve a patient's X-ray to compare it with previous scans and track treatment progress over time. The deleting files option 836 facilitates that outdated or incorrect documents can be removed to maintain accurate records and prevent confusion. For example, if a lab technician uploads an incorrect STL file for a 3D dental model, they can delete it and replace it with the correct version, ensuring that only the most relevant and up-to-date files are available for review. In an embodiment, the file management processor may include version control, which tracks changes to uploaded documents, allowing users to access previous versions if needed. Access permissions may also be enforced, ensuring that only authorized users—such as dentists, consultants, and labs—can view, modify, or delete specific files. By integrating these file management capabilities, the system enhances collaboration among dental professionals, reduces errors, and streamlines case workflows, ultimately improving the efficiency of treatment planning and execution.

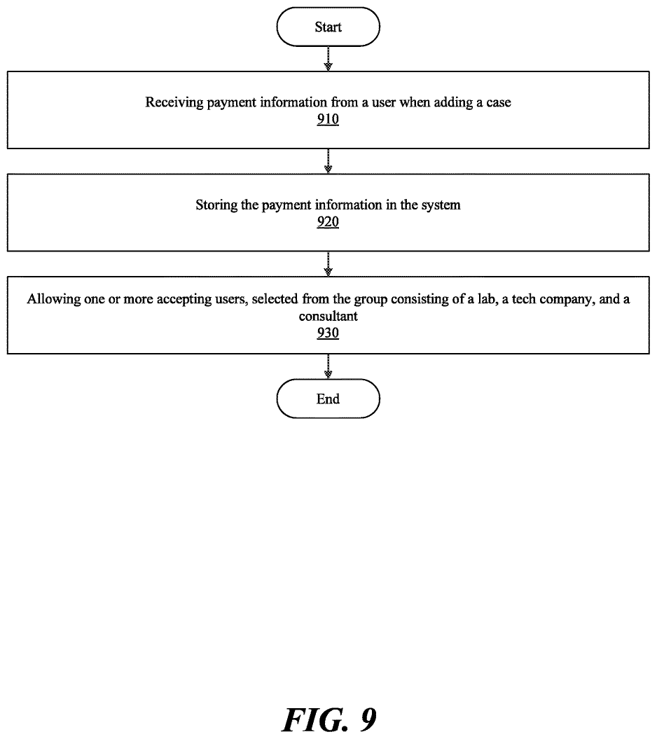

FIG. 9 is a flowchart describing a method for processing payment information within the case management system. At block 910, the system receives payment information from the user when a case is added. For example, a dentist may enter their billing details when creating a new case, ensuring transactions for associated services. This step may include selecting a preferred payment method, such as a credit card, bank transfer, or third-party payment provider. At block 920, the system securely stores payment information using encryption and compliance with industry security standards, such as PCI-DSS (Payment Card Industry Data Security Standard). For instance, a dental laboratory's payment details may be saved in the system for automated invoicing when they complete fabrication of a dental appliance. This secure storage facilitates that sensitive financial data remains protected. At block 930, the system allows accepting users, such as labs, tech companies, and consultants, to enter their payment information to facilitate transactions. A consultant offering remote treatment planning services, for instance, may input their bank details to receive payments for consultations. Similarly, a tech company providing AI-driven diagnostic tools may register their payment information to receive subscription-based fees from dental practices using their service. In some cases, users may also initiate payments when adding services to a case. For example, a dentist adding an orthodontic treatment plan to a patient's case may make an upfront payment to the dental lab for manufacturing clear aligners. Similarly, a consultant reviewing a complex case may require payment before approving a revised treatment plan. The system may further support automated invoicing, generating detailed breakdowns of charges for each case, and transaction tracking, which allows users to view payment history and pending transactions within the platform. By integrating these payment-processing capabilities, the system streamlines financial transactions, reduces administrative burden, and enhances collaboration among dental professionals and service providers.

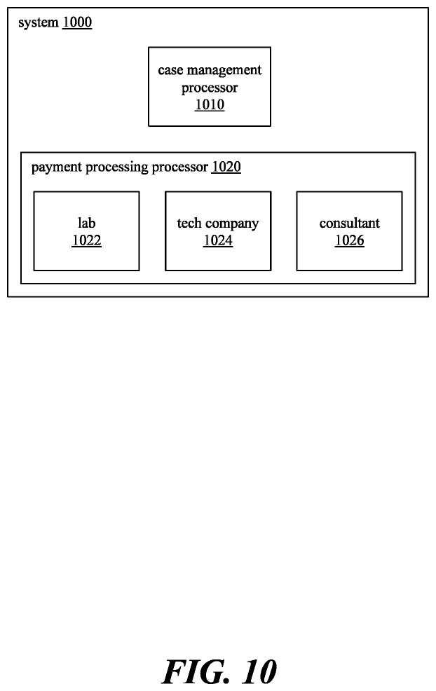

FIG. 10 is a block diagram of a system 1000, which details how payment processing is managed within the case management platform, ensuring financial transactions between users. The case management processor 1010 is responsible for receiving and securely storing payment information when a case is added. The payment processing processor 1020 may include entities such as a lab 1022, a tech company 1024, and a consultant 1026. The processor is responsible for receiving payments once the service has been completed. For example, when a dentist initiates a new patient case requiring orthodontic treatment, they can input their billing details and select a preferred payment method, such as a credit card, ACH transfer, or a third-party digital wallet. The system securely stores this data for future transactions, ensuring compliance with financial security standards such as PCI-DSS and HIPAA where applicable. The payment processing processor 1020 is configured to handle payments when services are added to a case. For instance, if a dental lab fabricates a custom crown, the system can automatically deduct payment from the dentist's stored payment method upon service completion. Similarly, if a consultant provides a case review or remote treatment planning, the system can process the transaction instantly, issuing an invoice and confirming payment receipt before granting the consultant access to the case details. The payment processing system can also facilitate split payments, ensuring that multiple service providers (e.g., a dental lab and a radiology service) receive their respective payments for contributions to a single case. In an embodiment, the system includes a refund processor that automates refunds when a service status is confirmed as incomplete or canceled. For example, if a dental lab fails to deliver a restoration due to manufacturing defects, the system can automatically initiate a partial or full refund to the dentist. Similarly, if a tech company providing AI-driven diagnostic services fails to generate an accurate report due to corrupted scan data, the system can adjust charges accordingly or issue a credit for future use. To enhance transparency, the system may also provide real-time transaction tracking, allowing users to monitor pending payments, completed transactions, and refund statuses. Notifications regarding successful payments, invoices, or refunds can be sent via email, SMS, or in-app alerts, ensuring all stakeholders remain informed about financial transactions related to their cases.

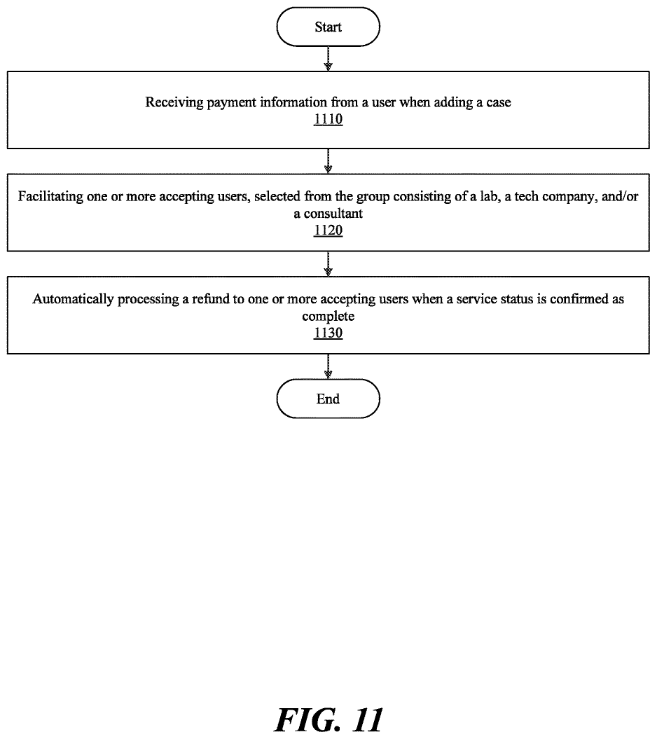

FIG. 11 is a flowchart describing a method for processing payment information in a case management system. At block 1110, the system receives payment information from a user when a case is added. For example, when a dentist initiates a new treatment case, they input their preferred payment method, such as a credit card, ACH transfer, or a third-party payment service like PayPal or Stripe. The system securely stores this information for future billing and transaction processing, adhering to financial security standards like PCI-DSS and HIPAA compliance to protect sensitive user data.

At block 1120, the system facilitates payments to accepting users, such as dental labs, tech companies, and consultants, once their services are completed. For instance, if a dental lab fabricates and delivers a custom crown, the system can automatically trigger payment from the dentist's stored payment method once the case status is updated to “completed”. Similarly, if a consultant provides a second opinion on a treatment plan, the system can release payment upon final approval by the dentist. The method also supports milestone-based payments, where partial payments are released as different phases of a service are completed—for example, an orthodontic case where payments are made after initial scans, mid-treatment adjustments, and final appliance delivery. At block 1130, the system automatically processes refunds when a service is confirmed as incomplete, canceled, or disputed. If a dental lab fails to deliver a prosthetic on time, the system may issue a full or partial refund back to the dentist. Similarly, if a tech company provides AI-generated diagnostics that are deemed inaccurate, the system can adjust the charges or provide a credit toward future services. In cases of disputes, the system may temporarily hold funds in escrow until a resolution is reached between parties. In an embodiment, users receive automated notifications regarding payment status, successful transactions, refunds, and invoices via email, SMS, or in-app alerts. The system may also generate detailed financial reports that allow users to track pending payments, completed transactions, and refund statuses, helping them manage their billing operations efficiently.

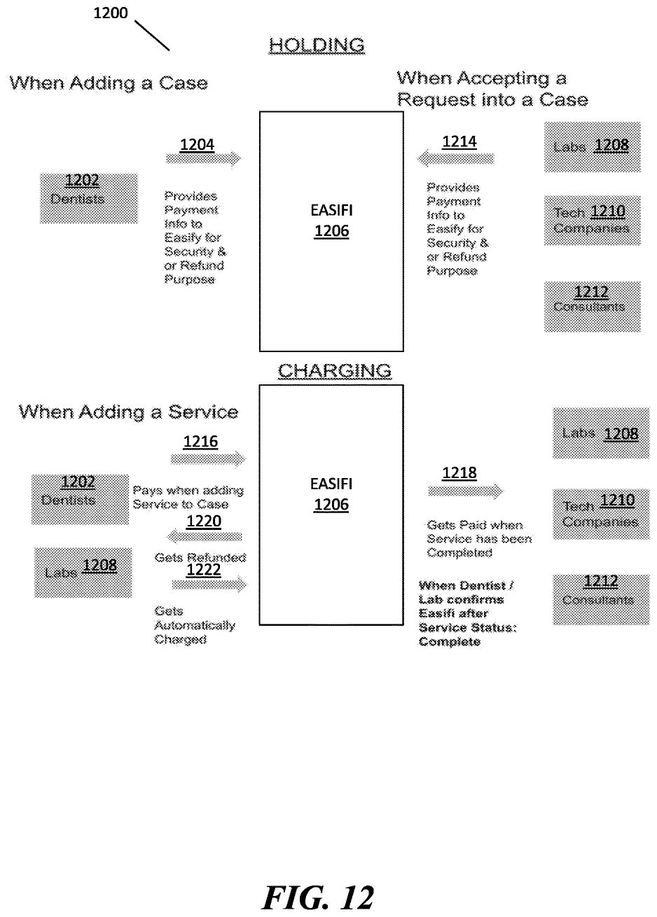

FIG. 12 illustrates a block diagram of the payment platform 1200 within the system. When adding a case, a user 1202 inputs payment information 1204 into the system 1206. One or more accepting users, such as a lab 1208, a tech company 1210, and/or a consultant 1212, also input payment information 1214 into the system 1206. When adding a service, a user 1202 makes a payment 1216 through the system 1206 at the time the case is added. Upon completion of the service, one or more accepting users, such as a lab 1208, a tech company 1210, and/or a consultant 1212, receive payment 1218 from the system 1206. In an embodiment, if necessary, one or more accepting users, such as a lab 1208, a tech company 1210, and/or a consultant 1212, may receive a refund 1220 from the system 1206. The system 1206 will automatically charge 1222 the accepting user when a user confirms with the system 1206 after the service status is marked as complete.

### EXAMPLES

As noted above, external users include Dentists, Consultants, Labs, and Tech Companies. Dentists are the primary users of the platform and are responsible for creating cases and managing the addition of other users and services to a case. Consultants, who are also dentists, have limited rights compared to Dentists. They assist Dentists on cases when assigned and can add services to a case, but they need the Dentist's approval because the Dentist is responsible for paying for those services. Labs are service providers added by either a Dentist or a Consultant. Once added, Labs can add services provided by other Labs or Tech Companies to a case, but they require the Dentist's approval. Labs are also responsible for paying for the services they add. Tech Companies are service providers added by either a Dentist or a Consultant but cannot add services to a case.

Internal users include Admins and Owners from Easifi, who can manage all external users, service providers, transactions, and the homepage of the platform, and the Super Admin from Medina Tech, who handles technical aspects. The platform's core features include authentication, case management, service management, file management, user profiles, search and filter functionality, sending and accepting requests, payment management, tracking case and service statuses, messaging, trash management, favorites, review and rating systems, and notifications. The project UI links for external users can be found in the provided Adobe XD links, while internal user interfaces are still in development. Some exceptions in the UI include an incomplete Tech Companies UI, pending updates for the Registration UI and Homepage UI, and a pending Payment UI.

For the project backlog, users, including Dentists, Labs, Consultants, and Tech Companies, should be able to register or sign up on the platform through navigation from the homepage. The registration fields differ slightly between users, with Dentists needing to provide more detailed information, such as NPI, License Number, and practice information, while Labs and Tech Companies only need basic contact details. Consultants' registration fields are similar to those of Dentists. The developers are advised to be mindful of the specific registration fields for each user type.

Users may authenticate themselves on the platform by logging in with their registered username and password. The platform allows users to reset their password via email or phone number and change their password through their profile, provided they input the correct old password. In an embodiment, users can log out of other devices if necessary, and Dentists will have their NPI and License Number verified during registration. Managing profiles is another feature. Users should be able to view and update their profile information, such as username, password, name, address, phone number, fax number, and email. Consultants, Labs, and Tech Companies can manage their public profiles, including listing services, adding business descriptions, providing training content, and displaying profile information on the About tab. Dentists, Labs, Tech Companies, and Consultants can navigate to another user's public profile via a hyperlink and send messages from that profile.

Case management allows Dentists to create, update, and delete cases. The case number is auto-generated, and Dentists must add mandatory fields like patient age, name, and address before creating a case. Only Dentists can view the patient's address. Dentists can change the case status to “Pending,” “In Progress,” or “Completed,” and they can add up to four services to a case. Consultants and Labs can add a maximum of three services, but they need approval from the Dentist. Services can only be added once they are marked as completed by the previous service provider. Dentists have the exclusive ability to delete a case. Case files can be uploaded, downloaded, and deleted by users who uploaded them. Dentists can delete any uploaded file. When managing case services, Dentists can add or cancel services but must send requests to service providers before adding them. Consultants and Labs can also add or remove services, but they must send requests to the service provider as well. All service changes must be confirmed by the Dentist before being sent to the service provider.

Users can manage their requests through the requests page, where they can accept or reject requests and cancel sent requests. They can also view pending requests, request history, and message other users. Requests for services are displayed under the Case Members section, with a default service status of “Pending.” The “All Cases” page allows users to create new cases, view and filter a list of cases, and search for past cases. The search and filter functionality facilitates users to sort cases by different criteria, such as ranking reviews, case type, service type, patient name, case status, and provider names. In an embodiment, users can manage reviews and ratings for services they have received. This feature is available for all users to facilitate transparency and quality control across the platform.

### Examples

The Admin has the ability to perform updates across various system pages, including the Contact Page, Privacy Policy, About Us, and other similar pages. In an embodiment, the Admin has the capability to access and view all user data, including the ability to view a comprehensive list of users and individual user profiles for detailed information. In terms of case management, the Admin has the ability to access and view a complete list of cases and review individual case details. The Admin also has control over payment methods, including the ability to view, update, and modify payment receive and sending methods. The Admin is authorized to process refunds for users as needed. Furthermore, the Admin has access to all invoice data, including the ability to view a complete list of invoices and specific invoices, and can update or generate new invoices, as well as modify their status.

When managing users, the Admin has control over enabling or disabling user accounts and making updates to individual user profiles. The Admin also has full control over case management, including the ability to edit case details and modify the statuses associated with cases. The Admin has the ability to send messages to all users. This includes viewing and managing the inbox, sending messages to other users from the inbox, and initiating messages to users from various parts of the application, similar to how users interact with the messaging system. In an embodiment, the Admin has control over all filter items, including the ability to view, enable, and disable filter options as necessary. The Admin also has the ability to manage deleted items. This includes viewing all items that have been moved to the trash, recovering them as needed, and configuring automatic deletion of trash items after a set period, ensuring the system remains organized and up-to-date.

### EXAMPLES

The distinguishing features of the system lie in its specific, non-generic implementation of payment processing and service management that is tailored to accommodate multiple user roles, such as labs, tech companies, and consultants. Unlike conventional systems that merely automate standard payment transactions, this system incorporates a dynamic payment structure that is condition-dependent, automatically triggering actions like refunds when a service status is completed and confirmed by a user. The system is not just performing generic functions of receiving and processing payments; it includes specialized logic for handling service-related transactions in real-time, based on specific conditions tied to each service's completion status. Moreover, the system is configured to handle multiple user roles and their respective actions in an automated manner. For instance, the lab, tech company, or consultant receives payment only when the corresponding service has been completed, and they are refunded automatically under specific conditions. This differs from generic systems, which may only handle basic payments without incorporating a sophisticated, status-dependent workflow.

In an embodiment, the interaction between the various components of the system, such as case management, payment processing, and refund issuance, is uniquely structured to facilitate that each user role has a distinct experience that is tailored to their function. The claims include specific, actionable steps like triggering payment confirmation only after a service status is updated, and issuing refunds based on user interaction. These technical aspects, such as the automated and conditional nature of the payment flows and the role-based user interaction, distinguish the system from conventional, generic computer systems performing generic tasks.

The system described includes features that distinguish it from a generic payment processing system. One of the primary distinguishing elements is its dynamic payment triggering mechanism. While most conventional systems allow users to make payments without any service-specific conditions, this system facilitates that payments are only processed after certain conditions are met. For example, a user may input payment information for a dental aligner case, but the system will only transfer the payment to the accepting user, such as a lab or consultant, once the service—like the creation or adjustment of the aligner, is marked as complete. This service-status-dependent payment process goes beyond the functionality of a generic system that merely processes transactions without taking service progress into account.

Another unique feature is the system's automated refund mechanism. Unlike a traditional system, where refunds are typically manual and disconnected from the service's performance, this system automatically triggers a refund if the service status is incomplete or erroneous. For instance, if a dental aligner lab completes the adjustment but the user confirms that the aligner does not meet the required specifications, the system will automatically issue a refund to the lab based on the specific service status and the user's confirmation. This dynamic relationship between service performance and refund issuance is more complex than the basic refund processes found in generic systems.

The system also introduces role-based payment reception, a feature not commonly seen in traditional payment systems. In this system, multiple accepting users, such as labs, tech companies, or consultants, are designated to receive payments based on the specific services they provide. For example, if a tech company is responsible for handling a case's software integration, it will receive payment once the user confirms that the service is complete. In contrast, a lab will only receive payment after producing the physical aligners. This nuanced, role-specific payment allocation distinguishes the system from generic systems that handle payments without considering the specific roles or services performed.

In an embodiment, the system automates charges based on user confirmations, which adds another layer of functionality not typically found in basic systems. For example, when a user confirms that a service, such as an aligner adjustment, has been completed to their satisfaction, the system automatically triggers the payment to the consultant or lab. This contrasts with traditional systems where payment processing often requires manual review or input, making the process more efficient and user-driven in this system.

Finally, the system facilitates complex user role interactions in payment and service management. In traditional systems, payments may flow in a simple, linear fashion, with one user making a payment to a single provider. However, this system tracks each service performed, assigns specific payments to the corresponding service providers, and manages refunds or charges based on service statuses. Multiple providers can be involved in a single case, with each provider receiving payment only after their specific service is completed and confirmed. This role-dependent payment structure is a departure from generic systems that do not consider the diverse and conditional nature of services provided by different parties. These features combine to make the system more advanced and specialized than generic payment systems, emphasizing automated, role-based payments and refunds, service status dependencies, and user-driven actions that are tailored to the specific needs of the dental and service industry context.

### Examples

This system improves the technical field by addressing challenges in payment processing, service tracking, and workflow automation, particularly for industries such as healthcare, consulting, and tech services. One of its primary innovations is the automated payment allocation tied to service completion. Unlike generic systems that process payments at initiation, this system facilitates that payments are only processed once specific tasks within a case are confirmed as completed. This feature reduces issues like premature payments and disputes over unfinished work, which is often seen in industries involving multi-step services, such as healthcare and consulting. In an embodiment, the system introduces a customizable payment workflow that adapts to the needs of different service providers. Whether a lab, a tech company, or a consultant is involved, each can define their service completion criteria, which automatically triggers the payment process. This level of customization allows the system to handle the complexities of multi-step services, something that generic systems fail to do effectively. The flexibility to tailor payment workflows to various service structures facilitates that payments are only released after a service provider meets specific conditions, streamlining operations and enhancing business processes.

The system also enhances coordination between multiple service providers involved in the same case. Unlike traditional systems where a user might interact with only one payment processor, this system facilitates that payments are allocated to multiple providers based on their respective tasks and completion statuses. This dynamic interaction makes it easier to manage cases involving multiple professionals, each responsible for different aspects of the service. It facilitates that the right provider is paid for their completed work in real-time, improving workflow efficiency. Moreover, the system integrates an automated refund mechanism that is triggered based on service status. If a service fails to meet predefined criteria, the system automatically processes refunds to the provider. This reduces the need for manual intervention and facilitates that refunds are issued only when services are not completed satisfactorily. This feedback loop, which is unique to the system, enhances customer satisfaction and facilitates that financial transactions are handled smoothly and transparently. Another notable feature is the granular payment tracking and reporting capabilities, which allow users to track payments at a detailed level. Each payment is linked to a specific service provider and task, offering transparency and accuracy in financial reporting. This feature is particularly valuable in industries where multiple services are rendered by different providers, and detailed payment histories are necessary for auditing, compliance, or dispute resolution.

The system also automates multi-stage services, where tasks performed by different providers trigger payments in sequence. This step-by-step automation facilitates that payments are processed at each stage of service completion, removing the need for manual tracking and reducing the chance of delays or errors. Furthermore, the system allows providers to set their own criteria for service completion, making it more adaptable to the diverse needs of various businesses. For example, a lab might set different criteria for service completion than a consultant, and the system facilitates that payments are processed according to these distinct requirements. This provider-specific customization enhances the accuracy of payment allocation and facilitates fairness in multi-provider service environments.

In addition, the system integrates real-time user feedback into the service completion process. Before triggering payment, users can rate or confirm the quality of the service, ensuring that payments are contingent on user satisfaction. This feature facilitates a more responsive and customer-centered approach to service delivery, which is particularly crucial in industries where service quality is paramount. The system also improves security and compliance, particularly for industries that deal with sensitive data, such as healthcare or consulting. By implementing features like data encryption, access control, and audit trails, the system facilitates that payment and service tracking data is handled securely and in compliance with industry regulations. This is especially important in sectors where privacy and data protection are concerns.

Lastly, the system's architecture is configured to scale efficiently, ensuring that it can handle large volumes of transactions and users without compromising performance. As the number of cases and service providers grows, the system maintains its functionality, ensuring smooth operation even in complex, high-volume environments. This scalability makes the system suitable for industries with varying service demands and transaction volumes. The system improves the technical field by introducing automation, flexibility, and scalability, which streamline service delivery, enhance payment accuracy, and facilitate secure, efficient operations. These features make it an invaluable tool for industries with multi-stage, multi-provider services, where tracking, payment, and service completion must be managed with precision and efficiency.

### Examples

Embodiment 1. A system for dental case management and collaboration, comprising: a web-based and mobile-based platform configured to facilitate communication and collaboration among dental professionals, laboratories, and third-party service providers; a presentation layer including a user interface for displaying case status, task lists, and patient information; an application layer including server-side logic for handling business processes, authentication, and role-based access control; a data layer including a database management system for storing one or more patient records, one or more treatment plans, or one or more transaction logs in a database; and a REST API configured to enable interoperability between the system and third-party service providers.
Embodiment 2. The system of embodiment 1, wherein the user interface is implemented using HTML5, CSS3, JavaScript, and React.js; and wherein the server-side logic is executed using Node.js and Express.js.
Embodiment 3. The system of embodiment 1, wherein the database is implemented using MongoDB for storing structured and unstructured dental case data.
Embodiment 4. The system of embodiment 1, further comprising an electronic signature feature for approving treatment plans digitally.
Embodiment 5. The system of embodiment 1, wherein the REST API allows integration with treatment planning software, remote monitoring systems, and payment processing services.
Embodiment 6. The system of embodiment 1, further comprising a billing and invoicing processor configured to process payments between dental professionals and service providers.
Embodiment 7. The system of embodiment 1, wherein patient records include 3D dental scans, intraoral images, and X-ray data stored in a secure format.
Embodiment 8. The system of embodiment 1, wherein case progress updates include color-coded status indicators for pending, in-progress, and completed stages.
Embodiment 9. The system of embodiment 1, wherein the platform supports multi-language interfaces for international accessibility.
Embodiment 10. A method for facilitating communication and collaboration in dental treatment planning, comprising: receiving patient case data from a dental professional via a web-based or mobile-based user interface; storing the patient case data in a database; processing the patient case data through server-side logic to generate treatment recommendations; providing access to third-party service providers via an API for consultation, remote monitoring, and treatment planning; and updating the user interface in real-time to display modifications, approvals, and case progress.
Embodiment 11. The method of embodiment 10, further comprising: automatically assigning one or more cases to dental laboratories based on predefined criteria.
Embodiment 12. The method of embodiment 10, wherein remote monitoring services provide real-time feedback on patient progress through wearable devices.
Embodiment 13. The method of embodiment 10, further comprising an automated case validation system that checks treatment plans against predefined clinical guidelines.
Embodiment 14. A cloud-based dental case management system, comprising: a cloud-hosted server configured to execute application logic and store patient case data; a user interface providing access to a treatment plan; a machine-learning algorithm configured to analyze patient treatment history and suggest optimized treatment paths; and an automated notification system configured to send alerts to dental professionals regarding case updates and deadlines.
Embodiment 15. The system of embodiment 14, wherein the system supports telehealth consultations between dental professionals and patients.
Embodiment 16. The system of embodiment 14, wherein the cloud-hosted server automatically scales based on a quantity of active cases and user load.
Embodiment 17. The system of embodiment 14, further comprising a role-based access control processor that grants different permission levels to dentists, laboratories, and consultants.
Embodiment 18. The system of embodiment 14, wherein the machine-learning algorithm is trained using historical patient treatment data to improve predictive accuracy.
Embodiment 19. The system of embodiment 14, wherein notifications are sent via SMS, email, and in-app alerts.
Embodiment 20. The system of embodiment 14, wherein the user interface providing access to an aligner design or a remote monitoring tool.

A computer readable storage medium may include any storage medium, or combination of storage media, accessible by a computer system during use to provide instructions and/or data to the computer system. Such storage media can include, but is not limited to, optical media (e.g., compact disc (CD), digital versatile disc (DVD), Blu-Ray disc), magnetic media (e.g., floppy disc, magnetic tape, or magnetic hard drive), volatile memory (e.g., random access memory (RAM) or cache), non-volatile memory (e.g., read-only memory (ROM) or Flash memory), or microelectromechanical systems (MEMS)-based storage media. The computer readable storage medium may be embedded in the computing system (e.g., system RAM or ROM), fixedly attached to the computing system (e.g., a magnetic hard drive), removably attached to the computing system (e.g., an optical disc or Universal Serial Bus (USB)-based Flash memory), or coupled to the computer system via a wired or wireless network (e.g., network accessible storage (NAS)).

Note that not all of the activities or elements described above in the general description are required, that a portion of a specific activity or device may not be required, and that one or more further activities may be performed, or elements included, in addition to those described. Still further, the order in which activities are listed are not necessarily the order in which they are performed. Also, the concepts have been described with reference to specific embodiments. However, one of ordinary skill in the art appreciates that various modifications and changes can be made without departing from the scope of the present disclosure as set forth in the claims below. Accordingly, the specification and figures are to be regarded in an illustrative rather than a restrictive sense, and all such modifications are intended to be included within the scope of the present disclosure.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims. Moreover, the particular embodiments disclosed above are illustrative only, as the disclosed subject matter may be modified and practiced in different but equivalent manners apparent to those skilled in the art having the benefit of the teachings herein. No limitations are intended to the details of construction or design herein shown, other than as described in the claims below. It is therefore evident that the particular embodiments disclosed above may be altered or modified and all such variations are considered within the scope of the disclosed subject matter. Accordingly, the protection sought herein is as set forth in the claims below.

What is claimed is:

1. A system for dental case management and collaboration, comprising:
a web-based and mobile-based platform configured to facilitate communication and collaboration among dental professionals, laboratories, and third-party service providers;
a presentation layer including a user interface for displaying case status, task lists, and patient information;
an application layer including server-side logic for handling business processes, authentication, and role-based access control;
a data layer including a database management system for storing one or more patient records, one or more treatment plans, or one or more transaction logs in a database; and
a REST API configured to enable interoperability between the system and third-party service providers.

2. The system of claim 1, wherein the user interface is implemented using HTML5, CSS3, JavaScript, and React.js; and
wherein the server-side logic is executed using Node.js and Express.js.

3. The system of claim 1, wherein the database is implemented using MongoDB for storing structured and unstructured dental case data.

4. The system of claim 1, further comprising an electronic signature feature for approving treatment plans digitally.

5. The system of claim 1, wherein the REST API allows integration with treatment planning software, remote monitoring systems, and payment processing services.

6. The system of claim 1, further comprising a billing and invoicing processor configured to process payments between dental professionals and service providers.

7. The system of claim 1, wherein patient records include 3D dental scans, intraoral images, and X-ray data stored in a secure format.

8. The system of claim 1, wherein case progress updates include color-coded status indicators for pending, in-progress, and completed stages.

9. The system of claim 1, wherein the platform supports multi-language interfaces for international accessibility.

10. A method for facilitating communication and collaboration in dental treatment planning, comprising:
receiving patient case data from a dental professional via a web-based or mobile-based user interface;
storing the patient case data in a database;
processing the patient case data through server-side logic to generate treatment recommendations;
providing access to third-party service providers via an API for consultation, remote monitoring, and treatment planning; and
updating the user interface in real-time to display modifications, approvals, and case progress.

11. The method of claim 10, further comprising:
automatically assigning one or more cases to dental laboratories based on predefined criteria.

12. The method of claim 10, wherein remote monitoring services provide real-time feedback on patient progress through wearable devices.

13. The method of claim 10, further comprising an automated case validation system that checks treatment plans against predefined clinical guidelines.

14. A cloud-based dental case management system, comprising:
a cloud-hosted server configured to execute application logic and store patient case data;
a user interface providing access to a treatment plan;
a machine-learning algorithm configured to analyze patient treatment history and suggest optimized treatment paths; and
an automated notification system configured to send alerts to dental professionals regarding case updates and deadlines.

15. The system of claim 14, wherein the system supports telehealth consultations between dental professionals and patients.

16. The system of claim 14, wherein the cloud-hosted server automatically scales based on a quantity of active cases and user load.

17. The system of claim 14, further comprising a role-based access control processor that grants different permission levels to dentists, laboratories, and consultants.

18. The system of claim 14, wherein the machine-learning algorithm is trained using historical patient treatment data to improve predictive accuracy.

19. The system of claim 14, wherein notifications are sent via SMS, email, and in-app alerts.

20. The system of claim 14, wherein the user interface providing access to an aligner design or a remote monitoring tool.
